(19) United States
(12) Patent Application Publication
(10) Pub. No.: US 2008/0262043 A1
(43) Pub. Date: Oct. 23, 2008

(54) Solid Crystalline Form of Pantoprazole Free Acid, Salts Derived Therefrom and Process for Their Preparation

(75) Inventor: Ignasi Auquer Pedemonte, Girona (ES)

Correspondence Address: HOGAN & HARTSON LLP;IP GROUP, COLUMBIA SQUARE, 555 THIRTEENTH STREET, N.W., WASHINGTON, DC, 20004, US

(73) Assignee: MEDICHEM, S.A., Barcelona, ES (ES)

(21) Appl. No.: 11/913,732

(22) PCT Filed: May 5, 2006
(86) PCT No.: PCT/IB2006/001968

§ 371 Date: Jun. 24, 2008

Related U.S. Application Data
(60) Provisional application No. 60/678,217, filed on May 6, 2005.

(30) Foreign Application Priority Data
May 5, 2006 (IB) PCT/IB2006/001968

(51) Int. Cl. A61K 31/4439; C07D 401/12

(52) U.S. Cl. 514/338; 546/273.7

(57) ABSTRACT

The invention relates, in general, to a new solid crystalline form of pantoprazole free acid (denominated “Form III”), salts derived therefrom (e.g., pantoprazole sodium and pantoprazole sodium sesquihydrate) and methods for producing the same. The invention further includes formulating pantoprazole free acid Form III, salts derived therefrom (e.g., pantoprazole sodium and pantoprazole sodium sesquihydrate) and/or in vivo cleavable prodrugs thereof (collectively “the compounds of the invention”) into readily usable dosage units for the therapeutic treatment (including prophylactic treatment) of mammals, including humans.

### CROSS REFERENCE TO RELATED APPLICATIONS

This application claims priority to U.S. Provisional Application No. 60/678,217, filed May 6, 2005, which is expressly incorporated herein by reference in its entirety.

### BACKGROUND OF THE INVENTION

1. Field of the Invention

The invention relates, in general, to a new solid crystalline form of pantoprazole free acid (denominated “Form III”), salts derived therefrom (e.g., pantoprazole sodium sesquihydrate) and methods for producing the same. The invention further includes formulating pantoprazole free acid Form III, salts derived therefrom (e.g., pantoprazole sodium sesquihydrate) and/or in vivo cleavable prodrugs thereof (collectively “the compounds of the invention”) into readily usable dosage units for the therapeutic treatment (including prophylactic treatment) of mammals, including humans.

2. Relevant Background

Pantoprazole (5-(difluoromethoxy)-2-[[(3,4-dimethoxy-2-pyridinyl)methyl]sulfinyl]-1H-benzimidazole) is a benzimidazole compound that inhibits gastric acid secretion. Pantoprazole sodium sesquihydrate has been approved by the FDA for parenteral administration under the name Protonix IV® and for oral administration under the name Protonix®, for short-term treatment of erosive esophagitis associated with gastroesophageal reflux disease (GERD), for maintenance of healing of erosive esophagitis and for the treatment of pathological hypersecretory conditions, including, for example, Zollinger-Ellison syndrome.

The pharmaceutically active ingredient pantoprazole is disclosed U.S. Pat. No. 4,758,579 (equivalent to EP 0 166 287), which characterizes pantoprazole only by its melting point.

According to WO 2004/080961, pantoprazole free acid exhibits polymorphism and is known to exist in at least in two crystalline forms (i.e., “Form I” and “Form II”) as well as in an amorphous form.

### SUMMARY OF THE INVENTION

The invention provides a new solid crystalline form of pantoprazole free acid (denominated “Form III”), salts derived therefrom (e.g., pantoprazole sodium sesquihydrate) and methods for producing the same. The invention further includes formulating pantoprazole free acid Form III, salts derived therefrom (e.g., pantoprazole sodium sesquihydrate) and/or in vivo cleavable prodrugs thereof (collectively “the compounds of the invention”) into readily usable dosage units for the therapeutic treatment (including prophylactic treatment) of mammals, including humans.

### BRIEF DESCRIPTION OF THE DRAWINGS

The accompanying drawings, which are included to provide a further understanding of the invention and are incorporated in and constitute a part of this specification, illustrate embodiments of the invention and, together with the description, serve to explain the principles of the invention. In the drawings:

FIG. 1 illustrates the X-ray powder diffractogram of pantoprazole free acid Form III obtained in Example 1/Step 1;

FIG. 2 illustrates the X-ray powder diffractogram of pantoprazole free acid Form III obtained in Example 1/Step 2;

FIG. 3 illustrates the X-ray powder diffractogram of pantoprazole free acid Form III obtained in Example 1/Step 3; and

FIG. 4 illustrates the superimposition of the X-ray powder diffractograms of the products obtained in Example 1/Steps 1-3.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein. In addition, and as will be appreciated by one of skill in the art, the invention may be embodied as a method, system or process.

The invention provides a new solid crystalline form of pantoprazole free acid (denominated “Form III”), salts derived therefrom (e.g., pantoprazole sodium sesquihydrate) and methods for producing the same.

A process for preparing a new polymorph of pantoprazole free acid (Form III), as illustrated in Scheme 1 below, comprises precipitating it from a mixture of ethyl acetate and water after the oxidation of 5-(difluoromethoxy)-2-[[(3,4-dimethoxy-2-pyridinyl)methyl]thio]-1H-benzimidazole with peracetic acid. Thereafter, the product is washed with basic water and is recrystallized twice in ethyl acetate to yield Form III pantoprazole free acid.

As further illustrated below in Scheme 1, Form III pantoprazole free acid can be converted to its corresponding salts including, pantoprazole sodium sesquihydrate.

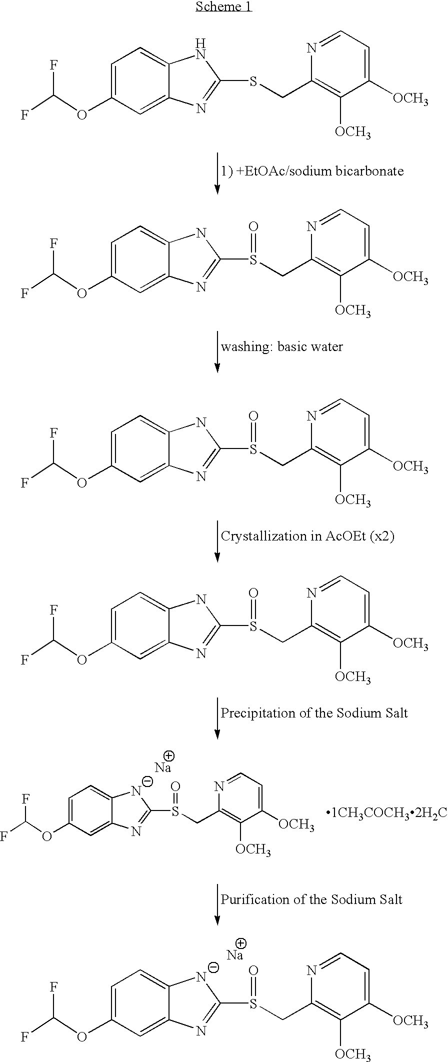

It has been further observed that there is no change in the crystalline structure of Form III pantoprazole free acid upon drying under vacuum at approximately 40° C. for approximately 2 hours.

The invention further includes formulating pantoprazole free acid Form III, salts derived therefrom (e.g., pantoprazole sodium sesquihydrate) and/or in vivo cleavable prodrugs thereof (collectively “the compounds of the invention”) into readily usable dosage units for the therapeutic treatment (including prophylactic treatment) of mammals, including humans. Such formulations are normally formulated in accordance with standard pharmaceutical practice as a pharmaceutical composition. According to this aspect of the invention, there is provided a pharmaceutical composition that comprises the compounds of the invention, as defined hereinbefore, in association with a pharmaceutically acceptable diluent or carrier.

The compositions of the invention may be in a form suitable for oral use (e.g., as tablets, lozenges, hard or soft capsules, aqueous or oily suspensions, emulsions, dispersible powders or granules, syrups or elixirs), for topical use (e.g., as creams, ointments, gels, or aqueous or oily solutions or suspensions), for administration by inhalation (e.g., as a finely divided powder or a liquid aerosol), for administration by insufflation (e.g., as a finely divided powder) or for parenteral administration (e.g., as a sterile aqueous or oily solution for intravenous, subcutaneous, or intramuscular dosing or as a suppository for rectal dosing). For example, compositions intended for oral use may contain, for example, one or more coloring, sweetening, flavoring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (e.g., polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (e.g., the sodium salt of benzoic acid, ethyl or propyl p-hydroxybenzoate), anti-oxidants (e.g., ascorbic acid), coloring agents, flavoring agents, and/or sweetening agents (e.g., sucrose, saccharine or aspartame).

Oily suspensions may be formulated by suspending the active ingredient in a vegetable oil (e.g., arachis oil, olive oil, sesame oil or coconut oil) or in a mineral oil (e.g., liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (e.g., sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insulation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used, and the aerosol device is conveniently arranged to dispense a metered quantity of active ingredient.

The amount of a compound of this invention that is combined with one or more excipients to produce a single dosage form will necessarily vary depending upon the host treated and the particular route of administration. For example, a formulation intended for oral administration to humans will may contain, for example, from 0.5 mg to 2 g of active ingredient compounded with an appropriate and convenient amount of excipients which may vary from about 5 to about 98 percent by weight of the total composition. Dosage unit forms will generally contain about 1 mg to about 500 mg of an active ingredient.

The size of the dose for therapeutic or prophylactic purposes of the compounds of the invention will naturally vary according to the nature and severity of the conditions, the age and sex of the animal or patient, and the route of administration, according to well known principles of medicine. For example, the method may comprise at least one of an hourly administration, a daily administration, a weekly administration, or a monthly administration of one or more compositions described herein.

In addition to the compounds of the invention, the invention also includes solvates, pharmaceutically acceptable prodrugs, pharmaceutically active metabolites, and pharmaceutically acceptable salts of such compounds.

The term “solvate” refers to an aggregate of a molecule with one or more solvent molecules.

A “pharmaceutically acceptable prodrug” is a compound that may be converted under physiological conditions or by solvolysis to the specified compound or to a pharmaceutically acceptable salt of such compound.

A “pharmaceutically active metabolite” is a pharmacologically active product produced through metabolism in the body of a specified compound or salt thereof. Metabolites of a compound may be identified using routine techniques known in the art, and their activities determined using tests such as those described herein.

Prodrugs and active metabolites of a compound may be identified using routine techniques known in the art. Various forms of prodrugs are known in the art.

According to the invention, suitable methods of administering the therapeutic composition of the invention to a patient include any route of in vivo administration that is suitable for delivering the composition into a patient. The preferred routes of administration will be apparent to those of skill in the art, depending on the type of condition to be prevented or treated, and/or the target cell population. Preferred methods of in vivo administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue.

It will be apparent to those skilled in the art that various modifications and variations can be made in the invention and specific examples provided herein without departing from the spirit or scope of the invention. Thus, it is intended that the invention covers the modifications and variations of this invention that come within the scope of any claims and their equivalents.

The following examples are for illustrative purposes only and are not intended, nor should they be interpreted to, limit the scope of the invention.

### Example 1

### Preparation of Pantoprazole Sodium Sesquihydrate via Pantoprazole Free Acid (Form III)

Step 1: Preparation of Wet Pantoprazole Free Acid (Form III)

A 2 L reactor equipped with a thermometer, mechanic agitation, an addition funnel and with a slight nitrogen overpressure was charged with 200 g (0.544 mol) of 5-(difluoromethoxy)-2-[[(3,4-dimethoxy-2-pyridinyl)methyl]thio]-1H-benzimidazole, 903 g (1000 mL) of ethyl acetate and 54.87 g (0.653 mol) of sodium bicarbonate to produce a yellowish suspension. The resulting mixture was cooled to approximately −1±2° C.

While holding the temperature of the mixture at approximately −1±2° C., the mixture was charged over 30 minutes with 90.63 mL (103.21 g, 0.493 mol) of peracetic acid (36.16% in acetic acid). An orange solution was observed when the addition was complete. The mixture was then maintained at approximately −1±2° C. for approximately 1 hour. Thereafter, while maintaining the temperature at approximately −1±2° C., 236.44 mL of sodium thiosulfate (4% in water; 0.0381 mol) was added over the course of 30 minutes. Next, in order to initiate precipitation, 200 mL of deionized water was added over 30 minutes while maintaining the temperature at approximately −1±2° C. The mixture was then seeded with approximately 1% of the raw 5-(difluoromethoxy)-2-[[(3,4-dimethoxy-2-pyridinyl)methyl]sulfinyl]-1H-benzimidazole). The mixture was then stirred at approximately −1±2° C. for approximately 3 hours and filtered to yield 320.0 g of wet pantoprazole acid (equivalent to 164.03 g of dry product, according to valuation; Yield: 78.59%). The wet pantoprazole acid (white solid) was washed with 1×150 mL of water (˜pH 8-9) and 2×75 mL of ethyl acetate. Chromatographic purity: 98.77% (area percent).

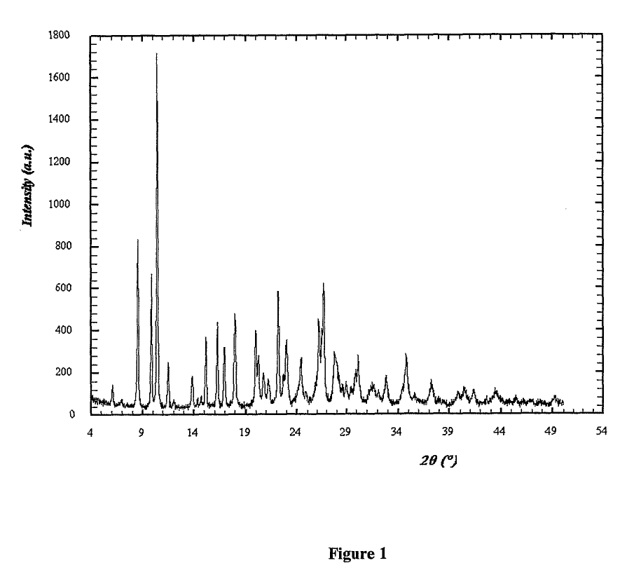

Analytical data: XRD (2θ): Main peaks at 8.53°, 9.86°, 10.44°, 15.13°, 16.26°, 16.95°, 17.98°, 19.94°, 20.19°, 22.21°, 22.98°, 24.36°, 26.19°, 26.70°, 27.81°, see FIG. 1.

Step 2: Washing of Pantoprazole Free Acid (Form III)

The wet pantoprazole acid obtained in the previous step is next washed with basic water (˜pH=12.2) using approximately 5 mL of water per gram of acid. The mixture was then stirred for 30 minutes at room temperature, and the solid was filtered and washed with water.

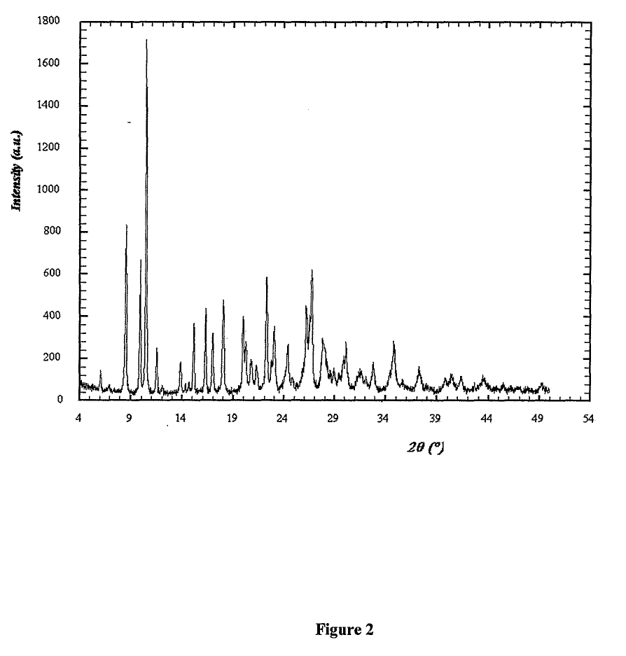

Analytical data: XRD (2θ): Main peaks at 8.51°, 9.84°, 10.42°, 15.11°, 16.25°, 16.93°, 17.97°, 19.94°, 20.18°, 22.19°, 22.96°, 24.31°, 26.21°, 26.66°, 27.85°, see FIG. 2.

Step 3: Recrystallization of Pantoprazole Free Acid (Form III)

The wet product obtained in the previous step (319.0 g wet pantoprazole acid equivalent to 163.51 g of dry product according to valuation) is next combined with 287.46 g of ethyl acetate. The mixture was then heated to approximately 37±2° C. for 30 minutes (maximum) to produce an orange solution. The mixture was next cooled to approximately 2±2° C. over 20 minutes and then stirred for 2 hours. The resulting product was filtered and washed with 2×12.4 mL of ethyl acetate to yield 299.18 g of wet 5-(difluoromethoxy)-2-[[(3,4-dimethoxy-2-pyridinyl)methyl]sulfinyl]-1H-benzimidazole (equivalent to 125.08 g of dried product; Yield: 76.5%). Chromatographic purity: target product, 99.50% (area percent).

Next, 298.0 g of the wet 5-(difluoromethoxy)-2-[[(3,4-dimethoxy-2-pyridinyl)methyl]sulfinyl]-1H-benzimidazole (equivalent to 124.59 g of dry product) was combined and crystallized from 164.10 g (181.73 mL) of ethyl acetate, as in the previous step, to yield 269.53 g of wet 5-(difluoromethoxy)-2-[[(3,4-dimethoxy-2-pyridinyl)methyl]sulfinyl]-1H-benzimidazole (equivalent to 120.77 g of dried product; Yield: 96.93%). Chromatographic purity: target product 99.60% (area percent).

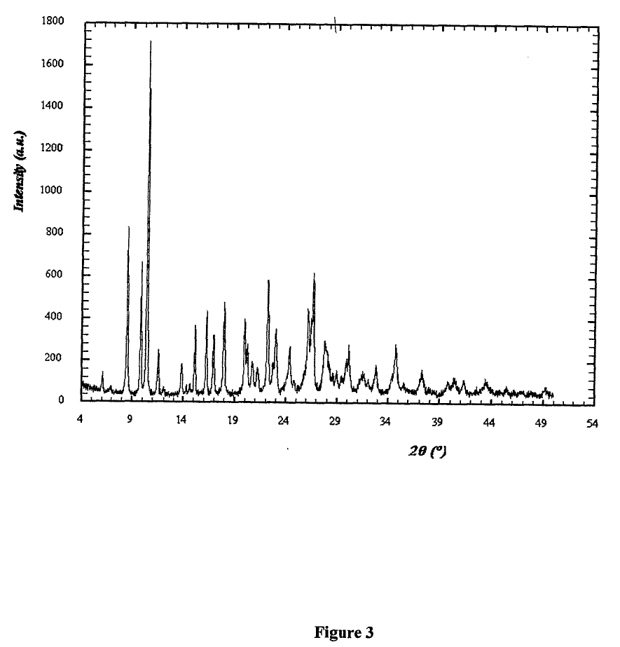

Analytical data: XRD (2θ): Main peaks at 8.50°, 9.83°, 10.42°, 15.10°, 16.23°, 16.93°, 17.96°, 19.92°, 20.17°, 22.18°, 22.96°, 24.33°, 26.19°, 26.66°, 27.73°, see FIG. 3.

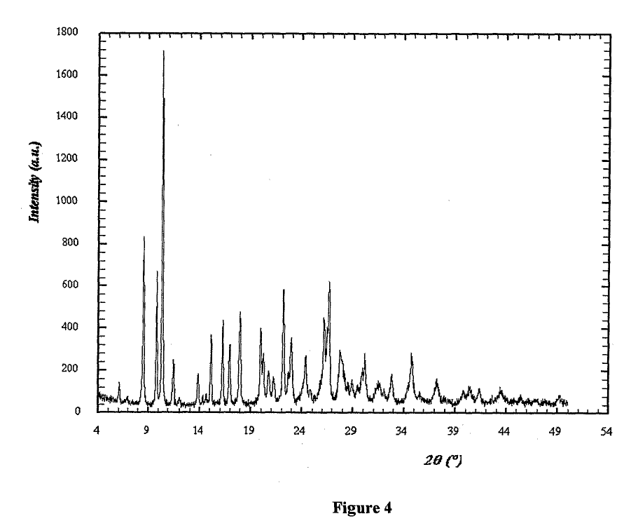

As illustrated in FIGS. 1 through 3, powder X-ray diffractograms of the solids isolated at each of the three preceding examples/steps demonstrates that the isolated solids correspond to a new polymorphic form of pantoprazole free acid (i.e., Form III). FIG. 4 illustrates the powder X-ray diffractograms of FIGS. 1 through 3 superimposed over one another and demonstrates that each corresponds to the new Form III polymorph of pantoprazole free acid.

Step 4: Preparation of Wet Pantoprazole Sodium Salt

The wet product obtained in the previous step (approximately 268.5 g) is combined with approximately 307.71 g (390 mL) of acetone and heated to approximately 28±2° C. to produce an orange solution. The orange solution was filtered to remove any insoluble particulates, and can optionally be treated with a decolorizing agent, to yield a clear orange solution. Thereafter, 24.13 g of a 52.8% solution of NaOH (0.305 mol, 1 eq.) was added over 10 minutes to produce an opalescent solution. Addition of the NaOH to the solution causes an exothermic reaction in which the temperature of the solution was observed to increase from 22° C. to 32° C. The mixture was next heated to approximately 37±2° C. to produce a transparent solution, which was subsequently cooled over 15 minutes to approximately 2±2° C. and then stirred at this temperature for approximately 2 hours. The mixture was then filtered and washed with 2×9.15 g (11.6 mL) of acetone to yield 169.36 g of wet 5-(difluoromethoxy)-2-[[(3,4-dimethoxy-2-pyridinyl)methyl]sulfinyl]-1H-benzimidazole) sodium salt (loss on drying: 29.91%; dry equivalent: 118.70 g). The product obtained was a complex of pantoprazole sodium salt with one molecule of acetone and two molecules of water. Chromatographic purity: target product, 99.80% (area percent).

Step 5: Preparation of Pantoprazole Sodium Salt Sesquihydrate

The sodium salt obtained in the previous step was purified by mixing 158.13 g of the obtained sodium salt with a mixture of 664.99 mL of ethyl acetate and 11.08 mL of water. The combined mixture was heated to approximately 37±2° C. to yield an orange-colored solution. Any acetone remaining from the previous step was then removed by distillation under vacuum at a moderated temperature (˜30° C.) to yield pantoprazole sodium salt sesquihydrate, which was then isolated by filtration.

Other processes according to the invention further include:

a. cooling a mixture of 5-(difluoromethoxy)-2-[[(3,4-dimethoxy-2-pyridinyl)methyl]thio]-1H-benzimidazole, ethyl acetate and sodium bicarbonate;
b. charging a solution of peracetic acid in acetic acid to the mixture;
c. stirring the mixture at low temperature;
d. adding sodium thiosulfate and water to the mixture;
e. filtering the precipitated 5-(difluoromethoxy)-2-[[(3,4-dimethoxy-2 pyridinyl)methyl]sulfinyl]-1H-benzimidazole) (i.e., pantoprazole free acid);
f. filtering and washing the solid;
g. suspending the solid in basic water and filtering;
h. crystallizing the solid in ethyl acetate and filtering;
i. optionally crystallizing the solid at least one additional time in ethyl acetate and filtering;
j. suspending the pantoprazole free acid in acetone and heating;
k. optionally, treating the solution with a decolorizing agent;
l. filtering the solution to remove insoluble particulates;
m. adding a sodium hydroxide solution;
n. heating the solution to approximately 37° C.;
o. cooling the solution to approximately −1° C.;
p. filtering the solid and washing with acetone;
q. suspending the solid in ethyl acetate and water;
r. heating the mixture to approximately 37° C. to obtain a solution;
s. removing approximately one half of the initial volume of solvent by distillation;
t. adding ethyl acetate to the solution;
u. cooling the solution;
v. filtering the solid; and
w. drying the solid under vacuum at approximately 30° C. to approximately 40° C. to obtain pantoprazole sodium sesquihydrate.

Although the invention has been described and illustrated with a certain degree of particularity, it is understood that the present disclosure has been made only by way of example, and that numerous changes in the conditions and order of steps can be resorted to by those skilled in the art without departing from the spirit and scope of the invention.

What is claimed is:

1. Pantoprazole free acid Form III and pharmaceutically acceptable salts derived therefrom.

2. The pantoprazole free acid Form III and pharmaceutically acceptable salts derived therefrom of claim 1, wherein said pantoprazole free acid Form III is characterized by an X-ray powder diffraction pattern (2θ) (±0.2°) having its main peaks at approximately 8.50°, 9.83°, 10.42°, 17.96°, 22.18° and 26.66°.

3. The pantoprazole free acid Form III and pharmaceutically acceptable salts derived therefrom of according to claim 2, further characterized by an X-ray powder diffraction pattern (2θ) (±0.2°) having peaks at approximately 15.10°, 16.23°, 16.93°, 19.92°, 20.17°, 22.96°, 24.33°, 26.19° and 27.73°.

4. The pharmaceutically acceptable salt of claim 1, wherein said salt is a sodium salt.

5. The sodium salt according to claim 4, wherein said salt is pantoprazole sodium sesquihydrate.

6. A pharmaceutical composition comprising at least one of pantoprazole free acid Form III of claim 1, pharmaceutically acceptable salts of said pantoprazole free acid Form III of claim 1 and combinations thereof and at least one of an adjuvant, a carrier, a diluent and another crystalline form of pantoprazole free acid or a pharmaceutically acceptable salt thereof.

7. The pharmaceutical composition of claim 6, wherein said salt of said pantoprazole free acid Form III is the sodium salt.

8. The pharmaceutical composition of claim 7, wherein said sodium salt of said pantoprazole free acid Form III is pantoprazole sodium sesquihydrate.

9. The pharmaceutical composition of claim 6, further comprising at least one of an adjuvant, a carrier, a diluent and another crystalline form of pantoprazole free acid or a pharmaceutically acceptable salt thereof.

10. A method of treating a mammalian condition comprising administering to a patient in need thereof a sufficient quantity of a composition comprising pantoprazole free acid Form III and pharmaceutically acceptable salts derived therefrom.

11. A process for preparing a pantoprazole sodium salt comprising converting pantoprazole free acid Form III into the pantoprazole sodium salt.

12. The process of claim 11 further comprising converting the pantoprazole sodium salt into pantoprazole sodium sesquihydrate.

13. A pharmaceutical composition comprising pantoprazole sodium sesquihydrate prepared according to claim 12.

14. A process for preparing pantoprazole free acid Form III comprising:
preparing a solution of 5-(difluoromethoxy)-2-[[(3,4-dimethoxy-2-pyridinyl)methyl]thio]-1H-benzimidazole and sodium bicarbonate in ethyl acetate;
adding peracetic acid to the solution to produce pantoprazole free acid Form III; and
isolating pantoprazole free acid Form III.

15. The process of claim 14, further comprising recrystallizing pantoprazole free acid Form III.

16. The process of claim 15, wherein said recrystallizing comprises recrystallizing in ethyl acetate.

17. The process of claim 14, wherein said isolating comprises precipitating pantoprazole free acid Form III.

18. The process of claim 17, wherein said precipitating step comprises seeding the solution with pantoprazole free acid Form III.

19. The process of claim 14, further comprising washing the pantoprazole free acid Form III with basic water.

20. The process of claim 19, wherein the basic water has a pH of approximately 12.2.

21. The process of claim 19, wherein said washing comprises washing with approximately 5 mL of basic water per gram of pantoprazole free acid Form III.

22. The process of claim 14, further comprising converting pantoprazole free acid Form III into a pantoprazole sodium salt.

23. The process of claim 22, further comprising converting the pantoprazole sodium salt to pantoprazole sodium sesquihydrate.

24. The process of claim 22, wherein said converting comprises:
heating a first solution of pantoprazole free acid Form III in acetone;
adding sodium hydroxide to the first solution and heating the combined solution; and
isolating the pantoprazole sodium salt from the combined solution.

25. (canceled)

26. The process of claim 24 further comprising purifying said pantoprazole sodium salt into pantoprazole sodium sesquihydrate.

27. The process of claim 24, wherein the first solution is heated to approximately 28±2° C.

28. The process of claim 24 further comprising at least one of (i) filtering the first solution and (ii) treating the first solution with a decolorizing agent.

29. (canceled)

30. The process of claim 24, wherein the combined solution is heated to approximately 37±2° C.

31. (canceled)

32. The process of claim 24 further comprising washing the pantoprazole sodium salt with acetone.

33. The process of claim 26, wherein said purifying comprises:
preparing a second solution of pantoprazole sodium salt, ethyl acetate and water;
heating the second solution;
distilling the second solution to remove any residual solvents; and
isolating pantoprazole sodium sesquihydrate from the second solution.

34. The process of claim 33 further comprising drying the pantoprazole sodium sesquihydrate.

35. The process of claim 33, wherein said second solution is heated to approximately 37±2° C.

36. The process of claim 34, where said drying step is carried out at a temperature of approximately 30° C.
